(19) United States
(12) Patent Application Publication
(10) Pub. No.: US 2025/0235180 A1
(43) Pub. Date: Jul. 24, 2025

(54) ULTRASOUND BEACON VISUALIZATION WITH OPTICAL SENSORS

(71) Applicant: DEEPSIGHT TECHNOLOGY, INC., Santa Clara, CA (US)

(72) Inventor: Danhua ZHAO, San Jose, CA (US)

(21) Appl. No.: 18/698,193

(22) PCT Filed: Oct. 7, 2022
(86) PCT No.: PCT/US2022/077762

§ 371 Date: Apr. 3, 2024

Related U.S. Application Data
(60) Provisional application No. 63/253,846, filed on Oct. 8, 2021.

(51) Int. Cl. A61B 8/08; A61B 5/06; A61B 8/00; G01S 7/52; G01S 15/89

(52) U.S. Cl. CPC A61B 8/0841; A61B 5/066; A61B 8/5207; G01S 7/5202; G01S 7/52028; G01S 7/52073; G01S 15/8915

(57) ABSTRACT

A method for visualizing position of an object may include emitting acoustic beamforming pulses and acoustic beacon pulses from an ultrasound array, receiving acoustic beamforming signals corresponding to the acoustic beamforming pulses and acoustic beacon signals corresponding to the acoustic beacon pulses with one or more optical sensors arranged on the object, generating an ultrasound image based on the acoustic beamforming signals, and generating an object indicator based on the acoustic beacon signals. The ultrasound image and the object indicator may be combined, such as for display.

### CROSS-REFERENCE TO RELATED APPLICATIONS

This application claims the benefit of U.S. Provisional Application No. 62/253,846, filed Oct. 8, 2021, the content of which is hereby incorporated by reference in its entirety.

### TECHNICAL FIELD

This invention relates generally to the field of visualizing and/or tracking objects using an ultrasound beacon signal.

### BACKGROUND

Acoustic imaging is used in various industries including medical imaging. For example, acoustic imaging technology may be used to visualize objects (e.g., needles, catheters, guidewires) used in clinical procedures such as biopsy, drug delivery, catheterization, device implantation, etc. Using acoustic imaging for medical applications offers several advantages. For instance, acoustic imaging such as ultrasound imaging is a non-invasive form of imaging. Additionally, ultrasound imaging uses ultrasound signals which are known to have remarkable penetration depth.

Some existing acoustic imaging technologies use piezoelectric (PZT) transducers to visualize and track objects (e.g., needles, catheters, drug delivery pumps, etc.). However, PZT transducers are generally limited by low output. Furthermore, imaging technology including PZT transducers often require bulky circuits. Therefore, it may be challenging to use PZT transducers for medical applications because of these size limitations (e.g., physical size). Accordingly, there is a need for new and improved compact technology with high sensitivity to visualize and track objects especially for medical applications.

### SUMMARY

Systems and methods for visualizing position of an object are described herein. In some variations, a method for visualizing position of an object may include emitting acoustic beamforming pulses and acoustic beacon pulses from an ultrasound array, receiving acoustic beamforming signals corresponding to the acoustic beamforming pulses and acoustic beacon signals corresponding to the acoustic beacon pulses with one or more optical sensors arranged on the object, generating an ultrasound image based on the acoustic beamforming signals, and generating an object indicator based on the acoustic beacon signals. The ultrasound array may comprise two or more transducers offset in a first dimension of the ultrasound array.

In some variations, emitting acoustic beacon pulses may comprise emitting a first acoustic beacon pulse from a first transducer and emitting a second acoustic beacon pulse from a second transducer. In some variations, the second transducer may be offset from the first transducer in the first dimension of the ultrasound array. In some variations, receiving the acoustic beacon signals may comprise receiving a first acoustic signal corresponding to the first acoustic beacon pulse and a second acoustic signal corresponding to the second acoustic beacon pulse with a single optical sensor in the one or more optical sensors.

In some variations, the method may further comprise receiving acoustic beamforming signals corresponding to the beamforming pulses with at least one transducer. In some variations, emitting beacon pulses may further comprise emitting a third acoustic beacon pulse from a third transducer. In some variations, the method may further comprise emitting the first acoustic beacon pulse from the first transducer at a first time and emitting the second acoustic beacon pulse from the second transducer at a second time subsequent to the first time.

The method may further comprise substantially simultaneously emitting the first acoustic beacon pulse from the first transducer and emitting the second acoustic beacon pulse from the second transducer. In some variations, the first acoustic beacon pulse may have a first transmit frequency and the second acoustic beacon pulse may have a second transmit frequency different from the first transmit frequency. In some variations, generating the object indicator may comprise filtering the received acoustic beacon signals into a first acoustic beacon signal corresponding to the first acoustic beacon pulse based on the first transmit frequency, and filtering the received acoustic beacon signals into a second acoustic beacon signal corresponding to the second acoustic pulse based on the second transmit frequency. In some variations, filtering the received acoustic beacon signals into the first and second acoustic beacon signals may comprise applying to the received acoustic beacon signals a comb filter having a first filtering band centered around the first transmit frequency and a second filtering band centered around the second transmit frequency.

In some variations, the first and the second transducers may be excited with different coded excitation parameters. In some variations, generating the object indicator may comprise applying a matched filter to decode the received acoustic beacon signals into a first acoustic beacon signal corresponding to the first acoustic beacon pulse and a second acoustic beacon signal corresponding to the second acoustic beacon pulse. The coded excitation parameters may comprise parameters forming orthogonal code pairs. In some variations, the orthogonal code pairs may be orthogonal Golay code pairs. In some variations, the coded excitation parameters may comprise parameters forming Barker code. In some variations, the coded excitation parameters may comprise parameters forming chirp code. In some variations, the coded excitation parameters may comprise parameters forming windowed nonlinear frequency modulation code.

In some variations, the method may further comprise alternating between emitting acoustic beamforming pulses and emitting acoustic beacon pulses. In some variations, the method may further comprise alternating between generating an ultrasound image based on the acoustic beamforming signals and generating an object indicator based on the acoustic beacon signals. In some variations, the method may further comprise substantially simultaneously emitting the acoustic beamforming pulses and the acoustic beacon pulses. The acoustic beamforming pulses may have a third transmit frequency and the acoustic beacon pulses have a fourth transmit frequency different from the third frequency. In some variations, the method may further comprise filtering the received acoustic beamforming signals based on the third transmit frequency, and filtering the received acoustic beacon signals based on the fourth transmit frequency.

In some variations, the method may comprise generating the object indicator comprises resolving the received acoustic beacon signals into a current object position. In some variations, the method may further comprise combining the ultrasound image and the object indicator. In some variations, one or more optical sensors may comprise an interference-based optical sensor. In some variations, one or more optical sensors may comprise an optical resonator or an optical interferometer. In some variations, the one or more optical sensors may comprise a whispering gallery mode (WGM) resonator.

In some variations, the one or more transducers may comprise a piezoelectric sensor, a single crystal material sensor, a piezoelectric micromachined ultrasound transducer (PMUT) sensor, or a capacitive micromachined ultrasonic transducer (CMUT) sensor.

In some variations, the first dimension is an elevation dimension of the ultrasound array. In some variations, the first dimension is a lateral dimension of the ultrasound array.

A system for visualizing position of an object may comprise an ultrasound array, at least one optical sensor, and at least one processor. In some variations, the ultrasound array may comprise a plurality of transducers configured to emit acoustic beamforming pulses and acoustic beacon pulses. In some variations, the plurality of transducers may comprise two or more transducers offset in a first dimension of the ultrasound array. In some variations, at least one sensor may be arranged on the object and may be configured to detect acoustic beamforming signals corresponding to the acoustic beamforming pulses and acoustic beacon signals corresponding to the acoustic beacon pulses. In some variations, at least one processor may be configured to generate an ultrasound image based on the acoustic beamforming signals and an object indicator based on the acoustic beacon signals.

In some variations, the plurality of transducers may comprise a first transducer configured to emit a first acoustic beacon pulse and a second transducer configured to emit a second acoustic beacon pulse. The second transducer may be offset from the first transducer in the first dimension of the ultrasound array. The plurality of transducers may comprise a third transducer configured to emit a third acoustic beacon pulse. A distance between the first transducer and the second transducer in a second dimension of the ultrasound array may be different from a distance between the third transducer and the second transducer in the second dimension of the ultrasound array. In some variations, the optical sensor may be an interference-based optical sensor. In some variations, the optical sensor may be an optical resonator or an optical interferometer. In some variations, the optical sensor may be a whispering gallery mode (WGM) resonator.

In some variations, the plurality of transducers may comprise one or more of a piezoelectric sensor, a single crystal material sensor, a piezoelectric micromachined ultrasound transducer (PMUT) sensor, and a capacitive micromachined ultrasonic transducer (CMUT) sensor. At least one processor may be further configured to combine the ultrasound image and the object indicator. In some variations, the system may further comprise a display configured to display one or more of the ultrasound image and the object indicator.

In some variations, the ultrasound array may be arranged on the object. In some variations, at least one optical sensor may be coupled to the object. In some variations, at least one optical sensor may be integrally formed with the object. In some variations, the object may comprise an elongate member and a distal end. In some variations, at least one optical sensor may be arranged on the distal end of the object. In some variations, at least one optical sensor may be arranged on the elongate member of the object. In some variations, the two or more optical sensors may be arranged on the elongate member of the object. In some variations, the object may comprise a needle. In some variations, the first dimension may be an elevation dimension of the ultrasound array. In some variations, the second dimension may be a lateral dimension of the ultrasound array. In some variations, the first dimension may be transverse to the second dimension.

### BRIEF DESCRIPTION OF THE DRAWINGS

FIG. 1 is an exemplary variation of a system for ultrasound visualization of an object.

FIG. 2A illustrates a portion of an exemplary variation of a system with a needle and an optical sensor attached to the needle to track the needle and/or determine a position of the needle.

FIG. 2B illustrates a portion of an exemplary variation of a system with a needle and two optical sensors attached to the needle to track the needle and/or determine a position of the needle.

FIG. 3 illustrates an exemplary variation of a 1.5D array including three elements configured to emit acoustic beacon pulse(s).

FIG. 4 illustrates an exemplary variation of a 1.5D array including three elements configured to emit acoustic beacon pulse(s).

FIG. 5 is an exemplary schematic illustrating positions of elements configured to emit acoustic beacon pulses and illustrating a position of an optical sensor in a Cartesian coordinate system so as to locate an object.

FIG. 6 is an exemplary variation of a system for ultrasound beacon visualization of an object.

FIG. 7 illustrates an exemplary variation of a transmitter.

FIG. 8 illustrates a portion of an exemplary variation of a system for ultrasound beacon visualization to receive signals and generate an ultrasound image and an object indicator.

FIG. 9 is a flow diagram illustrating an exemplary variation of a method for object tracking and visualization.

FIG. 10 is a flow diagram illustrating an exemplary variation of a needle visualization mode of operation.

FIG. 11A illustrates an exemplary variation of an acoustic beacon signal spectrum and an acoustic beamforming signal spectrum occurring simultaneously.

FIG. 11B illustrates filter responses of an exemplary variation of dual filters to separate the acoustic beacon signal and the acoustic beamforming signal shown in FIG. 11A.

FIG. 12 illustrates an exemplary variation of a comb filter with three distinct frequency sub-bands.

FIG. 13 illustrates an exemplary schematic of a combined ultrasound image and object indicator.

### DETAILED DESCRIPTION

Non-limiting examples of various aspects and variations of the invention are described herein and illustrated in the accompanying drawings.

Systems, devices, and methods for ultrasound beacon visualization with optical sensors are described herein. For example, the technology described herein may track and monitor objects during medical procedures using acoustic beacon signals with optical sensors. The technology described herein may be compact in size and have high sensitivity, thereby improving visualization for medical applications such as medical imaging for tracking objects (e.g., needle, catheter, guidewire) during biopsy, drug delivery, catheterization, combinations thereof, and the like.

Object visualization in medical applications may be an important component for performing a medical procedure in a safe and reliable manner. For instance, a medical practitioner may visualize and track a needle tip while administering anesthesia to ensure safety. In such instances, adequate needle tip visualization may reduce and/or prevent unintentional vascular, neural, or visceral injury. Similarly, it may be helpful to visualize needles when performing medical procedures such as Seldinger technique or catheterization that facilitate access to blood vessels and/or other organs in a safe and consistent manner.

There are several drawbacks associated with conventional ultrasound imaging technologies for medical applications. For example, traditional ultrasound may use imaging probes configured to emit ultrasound waves, but due to the smooth surface of a needle, the incident ultrasound waves reflected from the needle surface may be steered away from an ultrasound receiver, thus weakening the deflection of the reflected waves. Piezoelectric (PZT) transducers are conventionally placed at the tip of a needle and configured to emit ultrasound waves for tracking the needle tip. However, PZT transducers have low output and low sensitivity due to their size requirements. PZT transducers may need bulky circuits for visualization, thereby limiting their application for medical procedures.

In contrast, the systems and devices described herein may be compact in size and have high sensitivity. In some variations, a method for visualizing a position of an object (e.g., end effector such as a needle, catheter, drug delivery pump) may use an ultrasound array including one or more optical sensors arranged on the object (e.g., coupled to the object, integrally formed with the object). In some variations, the ultrasound array may include two or more transducers in a first dimension (e.g., predetermined direction, elevation dimension, lateral dimension) of the ultrasound array and the optical sensor(s). For example, the elevation dimension may correspond to the y-axis and the lateral direction may correspond to the x-axis of a Cartesian coordinate system. For example, the transducers in the ultrasound array may be arranged such that two or more transducers may be spaced apart (e.g., offset) from each other in at least a first dimension (e.g., separated from each other by a predetermined elevation relative to ground, separated by different distances to a midline of a lateral dimension). In some variations, these two or more transducers may be offset from a center of the ultrasound array (e.g., in a first dimension). The method may include emitting acoustic beamforming pulses and acoustic beacon pulses from an ultrasound array. Acoustic beamforming signals corresponding to the acoustic beamforming pulses may be received with one or more optical sensors. Acoustic beacon signals corresponding to the acoustic beacon pulses may be received with one or more optical sensors. An ultrasound image may be generated based on the acoustic beamforming signals. Additionally or alternatively, an object indicator (e.g., graph, trace, grid, visual indicator) may be generated based on the acoustic beacon signals. The object indicator may be representative of the current position of the object. Furthermore, the current position of the object may be stored/or tracked over time to facilitate display and/or other visualization of the object's position and/or trajectory.

### Exemplary Systems

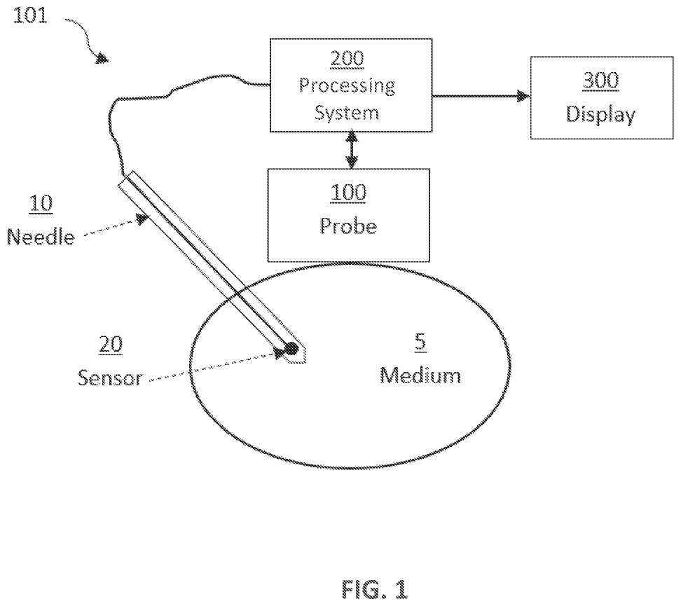

FIG. 1 is an exemplary variation of a system 101 for ultrasound visualization of an object. System 101 may be used for ultrasound beacon visualization of a needle 10; however, it should be understood that in other variations the system 101 may be used for ultrasound visualization of other objects (e.g., end effectors) such as a catheter, a guidewire, an endoscope, a trocar, an implant, combinations thereof, and the like. In some variations, the system 101 may comprise a processing system 200 coupled to a probe 100, one or more end effectors 10 (e.g., needle), and an optional display 300. In some variations, the end effector 10 may comprise one or more sensors 20, where the end effector 10 may be advanced into a medium 5 (e.g., body tissue, body cavity). In some variations, a processing system 200 may be operably coupled to the probe 100 and the needle 10. The needle 10 may be configured to generate electrical signals configured to excite a set of transducers of the sensor 20 of the probe 100. The transducers (e.g., ultrasound array) in the probe 100 may be configured to emit acoustic beamforming pulses and/or acoustic beacon pulses toward a medium 5. In some variations, the medium 5 may comprise a non-linear medium such as for example, a body tissue. One or more optical sensors 20 arranged on at least a part of the end effector may be configured to receive acoustic beacon signals corresponding to the acoustic beacon pulses emitted by the transducers of the probe 100. The acoustic beacon signals may comprise optical signals that may be transmitted to the processing system 200 via an optical fiber or other suitable waveguide. In some variations, the probe 100 may be configured to receive acoustic beamforming signals reflected in response to interactions of the acoustic beamforming pulses with the medium 5 and/or the needle 10. In some variations, the probe 100 may be configured to transmit the received acoustic beamforming signals to the processing system 200. The processing system 200 may be configured to generate ultrasound images based on the received acoustic beamforming signals. The processing system 200 may be configured to analyze the optical signals to generate an object indicator corresponding to a location of the end effector 10. The ultrasound images and the object indicator may be optionally displayed on a display 300. Additionally or alternatively, the object indicator may be output as one or more of an audio signal and a haptic signal.

Although the object in FIG. 1 is shown to be a needle 10, it should be readily understood that any suitable object (e.g., end effector) may be visualized and/or tracked using the system 101. For example, system 101 may be used to visualize and/or track a set of objects including a catheter as it is being advanced into a blood vessel and/or organ.

### Probe

Generally, a probe 100 of a system 101 may be configured to couple to a medium (e.g., placed externally over body tissue) to emit and receive ultrasound signals. In some variations, the probe 100 may include an ultrasound array with one or more elements (e.g., transducers) to output (e.g., generate) acoustic pulses and/or receive acoustic signals (e.g., echo signals) corresponding to the acoustic pulses. For example, the ultrasound array may include one or more elements (e.g., transducers) configured to emit a set of acoustic beamforming pulses (e.g., ultrasound signals) and/or receive a set of acoustic beamforming signals (e.g., ultrasound echoes) corresponding to the set of acoustic beamforming pulses. Furthermore, the probe 100 may also include one or more elements (e.g., transducers) configured to emit a set of acoustic beacon pulses. While in some variations only optical sensor(s) may be configured to receive a set of acoustic beacon signals (e.g., ultrasound echoes) corresponding to the set of acoustic beacon pulses, in some variations one or more transducers may additionally or alternatively be configured to receive a set of acoustic beacon signals corresponding to the set of acoustic beacon pulses. The set of beamforming signals that correspond to the set of beamforming pulses may be used to generate ultrasound images. In some variations, a set of beacon signals that correspond to a set of emitted beacon pulses may be used for object tracking. For example, as discussed above, a set of beacon signals may be converted into a set of optical signals that may be analyzed to determine a location of the object and/or to generate an object indicator.

In some variations, the elements of the probe 100 may be arranged as an array such as an ultrasound array. For example, probe 100 may include one or more transducers such as one or more of a piezoelectric transducer, a lead zirconate titanate (PZT) transducer, a polymer thick film (PTF) transducer, a polyvinylidene fluoride (PVDF) transducer, a capacitive micromachined ultrasound transducer (CMUT), a piezoelectric micromachined ultrasound transducer (PMUT), a photoacoustic transducer, a transducer based on single crystal materials (e.g., LiNbO₃(LN), Pb(Mg_{1/3}Nb_{2/3})—PbTiO₃ (PMN-PT), and Pb(In_{1/2}Nb_{1/2})—Pb(Mg_{1/3}Nb_{2/3})—PbTiO₃ (PIN-PMN-PT)), combinations thereof, and the like. It should be understood that the probe 100 may include a plurality of any of the transducer types. In some variations, the ultrasound array may include the same type of elements. Alternatively, the ultrasound array may include different types of elements. Additionally, in some variations, the ultrasound array may include one or more optical sensors, such as an interference-based optical sensor, which may be one or more of an optical interferometer and an optical resonator (e.g., whispering gallery mode (WGM) resonators).

In some variations, the probe 100 may comprise one or more housings (e.g., enclosures) with corresponding ultrasound arrays that may have the same or different configurations and/or functions. For example, different portions of the probe 100 may be placed externally over different portions of a tissue 5.

The ultrasound transducer arrays described herein may have various dimensionalities. For example, the array may be configured for operation in a 1 dimensional (1D) array configuration, a 1.25 dimensional (1.25D) array configuration, a 1.5 dimensional (1.5D) array configuration, a 1.75 dimensional (1.75D) array configuration, and a 2 dimensional (2D) array configuration, as described in more detail herein. Generally, dimensionality of an ultrasound transducer array relates to one or more of a range of an elevation beam width (e.g., elevation beam slice thickness), aperture size, foci, and steering throughout an imaging field (e.g., throughout an imaging depth).

In some variations, a 1D array may comprise only one row of elements in a first dimension (e.g., elevation dimension) and a predetermined (e.g., fixed) elevation aperture size. For example, a 1D array may comprise a plurality of array elements arranged in a single (e.g., only one) row extending in a single (e.g., first) dimension (e.g., the lateral dimension). In some variations of a linear array, a spacing between two adjacent elements may be equal to about one wavelength of a transmitted acoustic wave. In some variations of a phased array, a spacing between two adjacent elements may be about half a wavelength of a transmitted acoustic wave. Due to the single dimension of the 1D array, an elevation aperture size and elevation focus may both be fixed. Accordingly, a thin slice thickness in the elevation dimension cannot be maintained throughout the imaging depth.

In some variations, a 1.25D array may comprise a plurality of rows of elements in a first dimension (e.g., elevation dimension), a variable elevation aperture size, and a predetermined (e.g., fixed) elevation focal point via an acoustic lens. In some variations, the elevation aperture size may be electronically adjusted (e.g., varied, modified, controlled) to control (e.g., narrow) the elevation beam width and elevation beam slice thickness. In some variations, the elevation beam width may be reduced by adding more rows in the array in the elevation dimension. However, a 1.25D array has a predetermined elevation foci such that the beam thickness may not be controlled throughout an imaging field (e.g., imaging depth).

In some variations, a 1.5D array may comprise a plurality of rows of elements in a first dimension (e.g., elevation dimension), a variable elevation aperture size, and a variable elevation focus via electronical delay control. In some variations, a number of array elements may be larger than a number of channels in the imaging system. Moreover, one or more analog switches (e.g., high voltage switches) may be configured to select a set of sub-apertures of a 1.5D array. Accordingly, the 1.5D array may be configured to provide a relatively narrower elevation beam width throughout the imaging field, and enable imaging of smaller lesions at various imaging depths. For example, the 1.5 D array may include a relatively thinner elevation beam slice thickness for resolving smaller objects (e.g., blood vessels, cysts). Furthermore, the 1.5D array may provide more uniform image quality for near-field and far-field images.

In some variations, a 1.75D array may comprise a 1.5D array with additional elevation beam steering capability (e.g., up to about 5 degrees in at least one dimension, up to about 10 degrees in at least one dimension, up to about 15 degrees in at least one dimension, or up to about 20 degrees in at least one dimension).

In some variations, a 2D array may comprise a plurality of elements in a first dimension and a second dimension (e.g., both lateral and elevation dimensions) to satisfy a minimum pitch requirement for large beam steering angles. Like the 1.5D array, a system incorporating a 2D array may include one or more analog switches to select a predetermined set of sub-apertures of the array.

In some variations, the transducers of the ultrasound array may be spaced apart (e.g., offset) from each other in one or more dimensions (e.g., directions). For example, the array in the probe 100 may have an elevation characteristic that is greater than that of a 1D ultrasound array. For instance, in some variations, the array may be a 1.25D ultrasound array, a 1.5D ultrasound array, and/or a 2D ultrasound array.

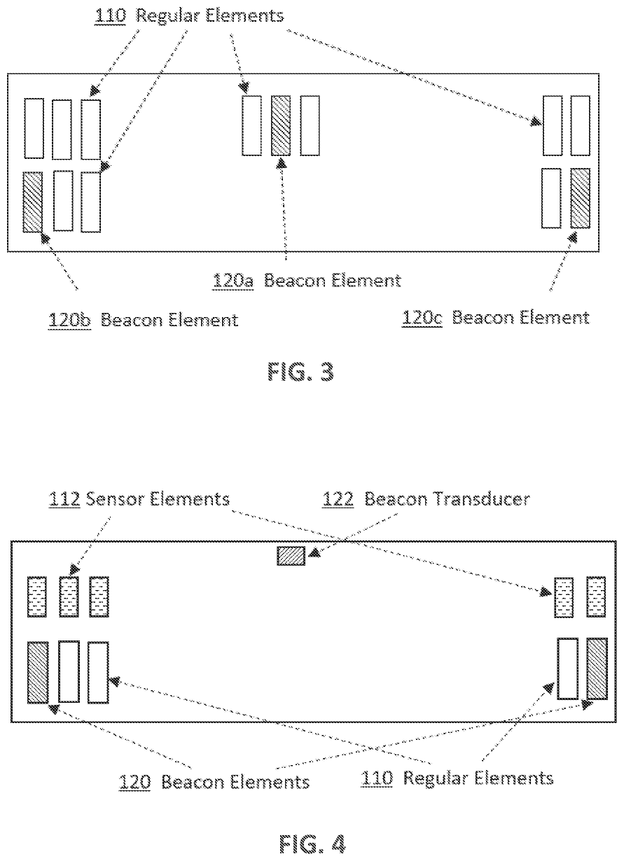

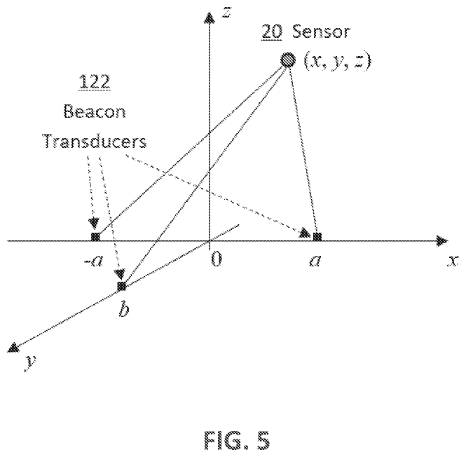

In some variations, one or more acoustic beacon pulse-emitting elements in the array may be offset in a first dimension (e.g., elevation dimension) of the array relative to the other acoustic beacon pulse-emitting element(s). In some variations, at least one acoustic beacon pulse-emitting element is located at a location to form a triangle with at least two of the other acoustic beacon pulse-emitting elements. In some variations, one or more elements in the first dimension may be configured emit acoustic beacon pulses. Additionally or alternatively, one or more elements in the first dimension may emit acoustic beamforming pulses. Additionally or alternatively, one or more elements in the first dimension may receive acoustic beamforming signals corresponding to the acoustic beamforming pulses. In some variations, the array may be a 1D array but may include at least one element that may be offset from the other elements in the array in a first dimension. In some variations, the at least one element may be offset from the center of the array. Further examples of 1.5D arrays are described in further detail below with respect to FIGS. 3 and 4. In some variations, the array in the probe 100 may be a phased array of elements.

### Example 1.5D Arrays

As discussed above, a probe (e.g., probe 100 in FIG. 1) may include an array of elements to generate acoustic beamforming pulses and acoustic beacon pulses and to receive acoustic beamforming signals. The array may have an elevation dimensionality that is greater than 1. In some variations, the array may be 1.5D array. The elements in the 1.5D array may be any suitable element or combination of elements such as piezoelectric transducers, CMUT transducers, PMUT transducers, transducers based on single crystal materials, a combination thereof, and/or the like.

In some variations, the same transducer elements may be used to emit the set of acoustic beamforming pulses, emit the set of acoustic beacon pulses, to receive the set of acoustic beamforming signals, and/or to receive the set of acoustic beacon signals (e.g., in combination with optical sensor(s) receiving acoustic beacon signals).

For example, a first set of transducers may be configured to emit both acoustic beamforming pulses and acoustic beacon pulses. In some variations, one or more transducers of the set of transducers may be configured to receive one or more of an acoustic beamforming signal and an acoustic beacon signal. Additionally or alternatively, a second set of transducers different from the first set of transducers may be configured to receive one or more of the acoustic beamforming signal and the acoustic beacon signal.

Alternatively, different transducers may be configured to emit acoustic beamforming pulses and acoustic beacon pulses. For example, a first set of transducers may be used to emit acoustic beamforming pulses and a second set of transducers may be used to emit acoustic beacon pulses. One or more transducers of the first and second set of transducers may be configured to receive one or more of an acoustic beamforming signal and an acoustic beacon signal. Additionally or alternatively, a third set of transducers different from the first and second sets of transducers may be configured to receive one or more of an acoustic beamforming signal and an acoustic beacon signals.

Transducer elements configured to emit acoustic beamforming pulses and/or acoustic beacon pulses may be excited in any suitable manner. For example, array elements may be excited in a manner such that acoustic beamforming pulses and acoustic beacon pulses are emitted in an alternating (e.g., interleaved) fashion. For instance, elements configured to emit acoustic beamforming pulses may be excited first, followed by elements configured to emit acoustic beacon pulses, after which elements configured to emit acoustic beamforming pulses may be excited again. In a similar manner, elements configured to emit acoustic beacon pulses may be excited, followed by elements configured to emit acoustic beamforming pulses, after which elements configured to emit acoustic beacon pulses may be excited again. Additionally or alternatively, the elements in the array may be excited in a manner such that the acoustic beamforming pulses and the acoustic beacon pulses may be configured to emit substantially simultaneously. In some variations, the acoustic beamforming pulses may have a different frequency than the acoustic beacon pulses where the different frequencies may be used to distinguish between corresponding acoustic beamforming signals and acoustic beacon signals.

In some variations, 1.5D arrays may include elements arranged in two or more rows (e.g., two, three, four, etc.). In some variations, each row of a 1.5D array may have the same number of elements. Alternatively, each row of a 1.5D array may have different number of elements. Two or more elements of a 1.5D array may be configured to emit acoustic beacon pulses (“beacon element”). One or more of these beacon elements may be offset in an elevation dimension from one or more other beacon elements. For example, a first beacon element may be offset in an elevation dimension from a second beacon element. In some variations, at least one beacon element may be located at a location to form a triangle with at least two of the other acoustic beacon pulse-emitting elements. Offset in beacon elements in the elevation dimension and/or offset in beacon elements in the lateral dimension of the array may, for example, help facilitate a triangulation algorithm for determining position of the object based on the acoustic beacon signals corresponding to the acoustic beacon pulses.

In some variations, each element configured to emit acoustic beacon pulses may emit a beacon pulse at a different frequency. For instance, a first element configured to emit acoustic beacon pulses may be excited at a first frequency and a second element configured to emit acoustic beacon pulses may be excited at a second frequency. For example, each element configured to emit acoustic beacon pulses may be excited with different coded excitation parameters. Some non-limiting examples of coded excitation parameters include parameters forming orthogonal code pairs, Barker code, chirp code, windowed nonlinear frequency modulation, combinations thereof, and the like.

In some variations, the elements configured to emit acoustic beacon pulses may be excited sequentially. For example, a first element configured to emit acoustic beacon pulses may be excited at a first time and a second element configured to emit acoustic beacon pulses may be excited at a second time after the first time. For example, if three elements in a 1.5D array are configured to emit acoustic beacon pulses, a first element may be excited at a first time, a second element may be excited at a second time subsequent to the first time, a third element may be excited at a third time subsequent to the second time, and the first element may be excited again at a fourth time subsequent to the third time. In some variations, the sequential excitation may occur in a periodic manner. For example, the first element, the second element, and the third element may be excited at periodic (e.g., regular) time intervals. In some variations, the elements configured to emit acoustic beacon pulses may be excited substantially simultaneously (e.g., at different frequencies).

In some variations, the elements configured to emit acoustic beacon pulses may be configured to emit acoustic beamforming pulses. In some variations, the elements configured to emit acoustic beacon pulses may also be configured to receive acoustic beamforming signals that correspond to acoustic beamforming pulses emitted from the array. Therefore, the elements configured to emit acoustic beacon pulses may enable the generation of ultrasound images in addition to the generation of object indicators.

FIG. 3 illustrates an exemplary variation of a 1.5D array including a set of three elements 120 configured to emit a set of acoustic beacon pulses. In FIG. 3, the 1.5D array comprises two rows of elements (e.g., top row and bottom row). In some variations, each row in the array may have a different number of elements. For example, in FIG. 3, the top row has more elements than the bottom row. Additionally, in FIG. 3, the top row comprises one beacon element 120a configured to emit an acoustic beacon pulse, and the bottom row comprises two elements 120b and 120c configured to emit an acoustic beacon pulse at opposite ends of the array.

In some variations, the beacon element 120a may be offset from the beacon elements 120b and 120c in a first dimension (e.g., elevation dimension). Additionally, the beacon element 120a may be offset from a midline (not shown) between the beacon elements 120b and 120c, such that the distance between the beacon element 120a and the beacon element 120b in a second dimension (e.g., lateral dimension) is different than the distance between the beacon element 120a and the beacon element 120c in the second dimension. A first distance between the beacon element 120a and 120b may be different from a second distance between the beacon element 120a and 120c. The other elements 110 in the top row and the bottom row may be configured to emit acoustic beamforming pulses and receive acoustic beamforming signals corresponding to the acoustic beamforming pulses. In some variations, beacon elements 120a, 120b, 120c may be configured to emit acoustic beamforming pulses and/or receive acoustic beamforming signals. Therefore, while elements 110 enable generation of only the ultrasound images, beacon elements 120a, 120b, 120c may enable generation of ultrasound images in addition to generation of object indicators. Although only three beacon elements are shown, it should be understood that in some variations, the array may include any suitable number of beacon elements. It should be understood that the first dimension may be in any direction, and the second dimension may be transverse (e.g., perpendicular) to the first dimension. For example, a first dimension may correspond to an elevation or lateral dimension and a second dimension may rotate circumferentially about the first dimension. Therefore, in some variations, the first dimension may be a lateral dimension and the second dimension may be corresponding elevation dimension.

FIG. 4 illustrates an exemplary variation of a 1.5D array including three beacon elements (e.g., beacon elements 120 and beacon element 122) configured to emit an acoustic beacon pulse. Beacon element 122 in FIG. 4 is shown to be offset from the beacon elements 120 in a first dimension (e.g., elevation dimension) of the array and offset from a midline (not shown) between the beacon elements 120 in a second dimension (e.g., lateral dimension) of the array. In some variations, beacon element 122 may be a beacon transducer configured to solely emit acoustic beacon pulses. In addition to emitting acoustic beacon pulses, beacon elements 120 may be configured to emit acoustic beamforming pulses and/or receive acoustic beamforming signals. Accordingly, while beacon elements 120 may contribute to the generation of ultrasound images (e.g., in addition to the generation of an object indicator by emitting beacon signals), beacon element 122 may contribute only to the generation of an object indicator. In some variations, element 122 may be a same type of element as elements 120 (e.g., all three may be PZT transducers). Alternatively, element 122 may be a different type of element from elements 120.

In FIG. 4, the 1.5D array includes two rows. The top row comprises the beacon element 122 dedicated solely to emit acoustic beacon pulse(s) and optical sensor elements 112. Optical sensor elements 112 may be configured to receive acoustic beamforming signals. In some variations, optical sensor elements 112 may include any suitable optical sensor described herein. The bottom row in FIG. 4 comprises transducer elements 110 configured to emit acoustic beamforming pulses and receive acoustic beamforming signals and beacon elements 120 configured to emit acoustic beacon pulses. In FIG. 4, beacon elements 120 are shown to be positioned at opposite ends of the array, but may alternatively positioned in any suitable location in the bottom row.

FIGS. 3 and 4 are described as 1.5D arrays solely for illustrative purposes. As discussed above, 1.5D arrays may have any suitable number of rows with any suitable number of elements. Furthermore, the ultrasound array may include mixed types of elements (e.g., optical sensors and non-optical sensors). Examples of suitable such mixed arrays are described in further detail in International Patent App. No. PCT/US2021/033715, which is incorporated herein by reference. Elements configured to emit acoustic beacon pulses may also be configured to emit acoustic beamforming pulses and/or receive acoustic beamforming signals. Alternatively, at least some elements configured to emit acoustic beacon pulses may be solely dedicated to emitting acoustic beacon pulses. Beacon elements configured to emit acoustic beacon pulses may be positioned at any suitable location in the array with at least one beacon element being offset in an elevation dimension of the array and/or offset from a midline between at least two other beacon elements in a lateral dimension of the array.

### Processing System

The processing system 200 may be configured to transmit electrical signals to excite one or more of the elements in the probe 100. Additionally, the processing system 200 may be configured to receive electrical signals corresponding to a representation of converted ultrasound echoes (e.g., set of beamforming signals) from the probe 100. The processing system 200 may be configured to process these electrical signals to generate a set of ultrasound images. The processing system 200 may also be configured to receive a set of optical signals corresponding to a set of beacon signals via an optical fiber of one or more optical sensors 20. The processing system 200 may be configured to process the set of optical signals to generate an object indicator and/or to determine a location of the object (e.g., needle 10).

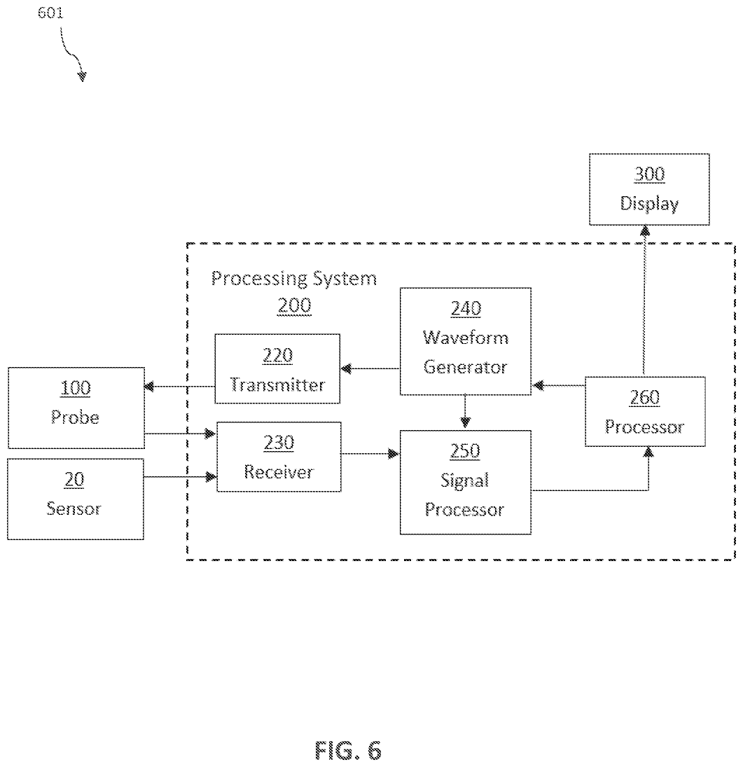

FIG. 6 illustrates another exemplary variation of a system 601 (e.g., structurally and/or functionally similar to system 101 in FIG. 1) for ultrasound beacon visualization of an object. FIG. 6 shows components of the processing system 200, according to some variations. For example, as seen in FIG. 6, the processing system may include a transmitter 220, a receiver 230, a waveform generator 240, and one or more processors (e.g., a signal processor 250 and processor 260). The waveform generator 240 may be configured to generate a set of digital waveforms for acoustic beamforming pulses and acoustic beacon pulses. One or more processors (e.g., processor 260) included in the processing system 200 may be configured to control the waveform generator 240. The waveform generator 240 may be configured to generate and send the digital waveforms to one or more of the transmitter 220 and/or a matched filter (not shown in FIG. 6).

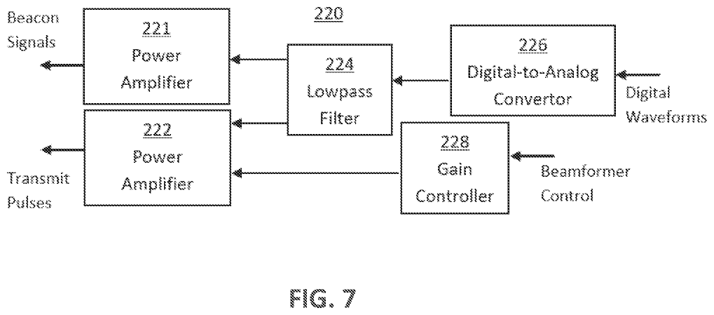

In some variations, the transmitter 220 may be configured to convert the set of digital waveforms into a set of electrical signals (e.g., high voltage electrical signals) configured to excite the elements in the ultrasound array of the probe 100. FIG. 7 illustrates an exemplary variation of a transmitter 220. In some implementations, the transmitter 220 may include one or more of a bipolar transmitter and a multilevel transmitter. It should be understood that the transmitter 220 may include a plurality of any of the transmitter types. In some variations, the transmitter 220 may include a set of custom-built transmitters (e.g., for generating continuous signals). As seen in FIG. 7, the transmitter 220 may comprise one or more of a Digital-to-Analog Converter (DAC) 226, a gain controller 228, a lowpass filter 224, and power amplifiers 221, 222. In some variations, the gain controller 228 may be omitted. The DAC 226 may be configured to convert digital waveforms into an analog signal. The lowpass filter 224 may be configured to smooth the analog signal. The gain controller 228 may be configured to transmit power (e.g., electrical signals) to excite elements in the probe 100 to emit acoustic beamforming pulses. In some variations, one or more of a set of transmit channels transmitting the electrical signals to the probe 100 may comprise the same drive voltage. Such variations may not need the gain controller 228. In some variations, a power amplifier 222 may be configured to adjust the voltage of the electrical signals for individual channels based on the output of the gain controller 228. For example, the power amplifier 221 may be configured to boost the amplitude of acoustic beacon pulses.

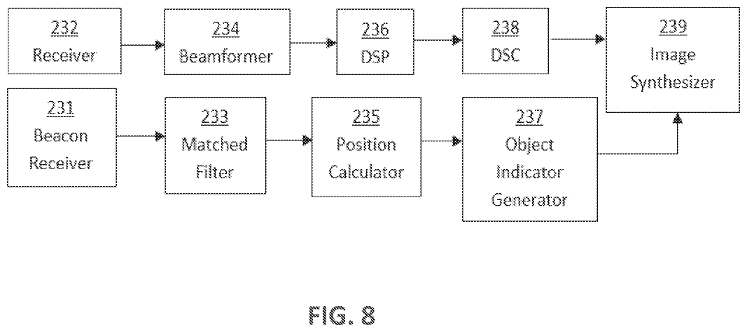

FIG. 8 illustrates an exemplary variation of at least some components of the processing system 200 configured to generate an ultrasound image and an object indicator. As seen in FIG. 8, the processing system 200 may include one or more of a receiver 232, a beamformer 234, a digital signal processor (DSP) 236 (e.g., signal processor 250 in FIG. 6), a digital scan converter (DSC) 238, an image synthesizer 239, a beacon receiver 231, a matched filter 233, a position calculator 235, and an object indicator generator 237.

In some variations, the receiver 232 may be configured to receive a set of beamforming signals (e.g., ultrasound echoes) from the probe 100. The receiver 232 may be configured to convert the beamforming signals (e.g., analog beamforming signals) into corresponding digital signals. The beamformer 234 may be configured to process the digitized beamforming signals received from the receiver 232. The DSP 236 may be configured to process the digitized beamforming signals by, for example, filtering, envelope detection, log compression, combinations thereof, and the like. The DSC 238 may be configured to convert individual scan lines generated following the processing of the digitized beamforming signals into a set of two-dimensional images.

In some variations, the beacon receiver 231 may be configured to receive the set of optical signals from an optical sensor 20. The beacon receiver 231 may be configured to convert the set of optical signals into a set of digital signals. In some variations, the matched filter 233 may be configured to process the digitized signals to maximize a signal-to-noise ratio. For example, the matched filter 233 may be configured to compress the set of digitized signals. The position calculator 235 may be configured to estimate the location of one or more of the optical sensors 20 as described in more detail below. In some variations, the object indicator generator 237 may be configured to generate an object indicator corresponding to a location of at least a part of the object (e.g., needle 10) (e.g., needle tip, needle body, etc.). The image synthesizer 239 may be configured to combine (e.g., overlay or otherwise merge) an ultrasound image and an object indicator to form a final display image.

As discussed above, one or more processors (e.g., signal processor 250, processor 260, etc.) included in the processing system 200 may be configured to perform one or more of data management, signal processing, image processing, waveform generation (e.g., beamforming, beacon, etc.), filtering, user interfacing, combinations thereof, and/or the like. The processor(s) may be any suitable processing device configured to run and/or execute a set of instructions or code, and may include one or more data processors, image processors, graphics processing units, digital signal processors, and/or central processing units. The processor(s) may be, for example, a general purpose processor, a Field Programmable Gate Array (FPGA), an Application Specific Integrated Circuit (ASIC), and/or the like. The processor(s) may be configured to run and/or execute application processes and/or other modules, processes and/or functions associated with the system 101.

In some variations, the processing system 200 may be configured to run and/or execute application processes and/or other modules. These processes and/or modules when executed by a processor may be configured to perform a specific task. These specific tasks may collectively enable the processing system 200 to transmit electrical signals to excite one or more elements of the probe 100, generate ultrasound images from beamforming signals, and generate object indicator from beacon signals. In some variations, application processes and/or other modules may be software modules. Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

In some variations, the processing system 200 may comprise a memory configured to store data and/or information. In some variations, the memory may comprise one or more of a random access memory (RAM), static RAM (SRAM), dynamic RAM (DRAM), a memory buffer, an erasable programmable read-only memory (EPROM), an electrically erasable read-only memory (EEPROM), a read-only memory (ROM), flash memory, volatile memory, non-volatile memory, combinations thereof, and the like. Some variations described herein may relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for the specific purpose or purposes.

### Display

In some variations, a display 300 may be configured to receive an output from the processing system 200. The display 300 may be operatively coupled to the processing system 200 and may be configured to display one or more of an ultrasound image (e.g., real-time ultrasound image) and one or more object indicators (e.g., graphic or other icon, trace, grid, visual indicators) representative of a position of an object. In some variations, the display 300 may be configured to display the ultrasound images and the set of object indicators in real time. In some variations, the set of object indicators may be overlayed with the ultrasound images. For instance, the ultrasound images may be displayed on the display 300 and the set of object indicators may be displayed over the ultrasound images on the display 300. The set of object indicators may be any suitable visual indicator representative of the position of the object (e.g., needle 10). For example, the set of object indicators may include a graphic that is positioned over the ultrasound image to represent the current position of the object relative to other objects (e.g., tissue features) in the ultrasound image. As such, the location of the object indicator(s) may communicate position within a field of view of the ultrasound probe.

As discussed above, the output from the processing system 200 may be sent to the display 300. A connection between the processing system 200 and the display 300 may be through a wired electrical medium (e.g., High Definition Multimedia Interface (HDMI), Digital Visual Interface (DVI), Video Graphics Array (VGA), and/or the like) and/or a wireless electromagnetic medium (e.g., WIFI™, Bluetooth®, and/or the like), and/or the like. The display 300 may be any suitable display such as liquid crystal display (LCD) monitors, organic light-emitting diode monitors (OLED), cathode-ray monitors (CRT), or any suitable type of monitor. In some variations, the display 300 may include an interactive user interface (e.g., a touch screen) and be configured to transmit a set of commands (e.g., pause, resume, and/or the like) to the processing system 200.

### Optical Sensor

As discussed herein, a set of acoustic beacon signals that correspond to a set of acoustic beacon pulses may be received by one or more optical sensors 20. In some variations, an optical sensor 20 may include one or more of an interference-based optical sensor, such as an optical interferometer, an optical resonator, and the like. Examples of optical interferometers include a Mach-Zehnder interferometer, a Michelson interferometer, a Fabry-Perot interferometer, a Sagnac interferometer, and the like. For example, a Mach-Zehnder interferometer may include two nearly identical optical paths (e.g., fibers, on-chip silicon waveguides, etc.) including finely adjusted acoustic waves (e.g., by physical movement caused by the acoustic waves, tuning of refractive index caused by the acoustic waves, etc.) to effect distribution of optical powers in an output(s) of the Mach-Zehnder interferometer, and therefore, detect a presence or a magnitude of the acoustic waves.

Additionally or alternatively, one or more of the optical sensors 20 may include an optical resonator. An optical resonator may include a closed loop of a transparent medium that allows some permitted frequencies of light to continuously propagate inside the closed loop, and to store optical energy of the permitted frequencies of light in the closed loop. For example, an optical resonator may be a whispering gallery mode (WGM) resonator, where the WGM resonator may permit propagation of a set of whispering gallery modes (WGMs) traveling a concave surface of the optical resonator where the permitted frequencies circulate the circumference of the optical resonator. Each mode from the WGMs may correspond to propagation of a frequency of light from the set of permitted frequencies of light. The set of permitted frequencies of light and the quality factor of the optical resonator may be based at least in part one or more of a set of geometric parameters of the optical resonator, refractive index of the transparent medium, and refractive indices of an environment surrounding the optical resonator.

In some variations, a WGM resonator may include a substantially curved portion (e.g., a spherical portion, a toroid-shaped portion, a ring-shaped portion). Furthermore, the substantially curved portion may be supported by a stem portion. The shape of a WGM resonator (e.g., the shape of the substantially curved portion of the WGM resonator) can be any suitable shape. For example, the shape of the WGM resonator can be spherical (e.g., a solid sphere), bubble shaped (e.g., spherical shape with a cavity), cylindrical, elliptical, ring, disk, toroid, and the like. Some non-limiting examples of WGM resonators include microring resonators (e.g., circular microring resonators, non-circular microring resonators such as resonators having a shape of racetrack, ellipse), microbottle resonators, microbubble resonators, microsphere resonators, microcylinder resonators, microdisk resonators, microtoroid resonators, combinations thereof, and the like.

Further examples of optical sensors (e.g., types of optical sensors, manufacturing and packaging of optical sensors) that may be used for beacon visualization are described in International Patent App. No. PCT/US2020/064094, International Patent App. No. PCT/US2021/022412, and International Patent App. No. PCT/US2021/039551, each of which is incorporated herein by reference.

### Input/Output Devices

In some variations, the system 101 may further include a set of input/output devices (not shown) configured to receive information input to the system 101 or output information from system 101. The set of input/output devices may include, for example, one or more of a keyboard, a mouse, a monitor, a webcam, a microphone, a touch screen, a printer, a scanner, a virtual reality (VR) head-mounted display, a joystick, a biometric reader, and the like. Additionally or alternatively, in some variations, the system 101 may include or be communicatively coupled to one or more storage devices (e.g., local or remote memory device(s)).

### End Effector

The optical sensor 20 may be arranged on (e.g., coupled to, mounted on, integrated with, or otherwise located on) at least a part of the end effector 10 (e.g., needle) to be tracked. In some variations, the end effector may include a needle 10 including a cylindrical body (e.g., barrel, tubing, lumen), an elongate member (e.g., plunger, shaft), and a distal tip. The elongate member may be configured to translate (e.g., slidably move) within the cylindrical body (e.g., the elongate member may translate within the cylindrical body). The elongate member may be coupled to any suitable actuation mechanism (e.g., actuator) configured to inject and/or withdraw fluid to and from the cylindrical body. For example, manually moving the elongate member within the cylindrical body may inject and/or withdraw fluid to and from the cylindrical body. Additionally or alternatively, the elongate member may be coupled to an actuator such as for example, a motor, to move the elongate member within the cylindrical body so as to inject and/or withdraw fluid to and from the cylindrical body. The cylindrical body may be open at one end and may taper into a distal tip (e.g., hollow tip) at the other end. In some variations, the tip of the needle 10 may include an attachment (e.g., connector) for a stem having a piercing tip configured to pierce through a predetermined medium (e.g., skin of a patient). In some variations, the stem may be slender so as to be narrower in diameter than the needle 10. The tip may be any suitable type of tip such as Slip-Tip®, Luer-Lok®, eccentric, etc.

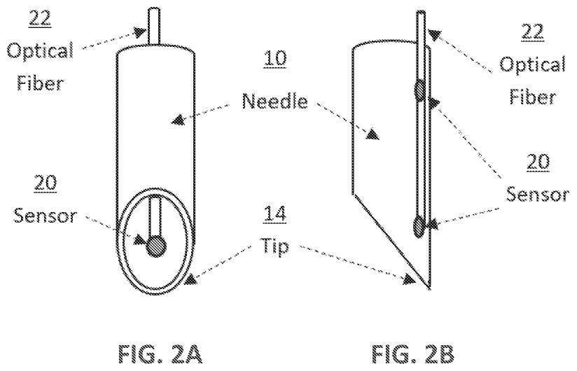

In some variations, the optical sensor may be arranged on (e.g., coupled to, mounted on, integrated with, or otherwise located on) the end effector 10 in any suitable manner, such as with epoxy or mechanical interfit features. FIG. 2A illustrates an exemplary variation of a system in which an optical sensor 20 is attached to a needle 10 to facilitate needle tracking and position determination. In FIG. 2A, the optical sensor 20 may be attached to, coupled to, integrated with, or otherwise mounted on a tip (e.g., distal tip) of the needle 10. The optical sensor 20 may be configured to detect acoustic beacon signals generated from a probe (e.g., probe 100 in FIG. 1). The optical sensor 20 may be configured to receive the acoustic beacon signals through a photo-elastic effect and/or a physical deformation of the optical sensor 20. For example, in the presence of acoustic beacon pulses, light, and/or sound waves (e.g., WGMs) received by the optical sensor 20 may undergo a spectral shift caused by changes in the refractive index and shape of the optical sensor 20. The optical sensor 20 may be configured to transmit a set of optical signals representative of the received acoustic beacon signals to a processing system (e.g., processing system 200 in FIG. 1). In some variations, the optical sensor 20 may be coupled to one or more optical waveguides 22 (e.g., optical fibers, photonic integrated circuit waveguides) to transmit the set of optical signals to the processing system. The processing system may be configured to generate an object indicator based on the optical signals. In some variations, the object indicator may be representative of a position of the tip of the needle 10 and/or may be used to track the tip of the needle 10. For example, the tip of the needle 10 may be visualized and tracked based on the object indicator. Accordingly, a needle 10 may be reliably visualized and tracked during a medical procedure using at least a single optical sensor 20.

FIG. 2B illustrates a cross-sectional view of an exemplary variation of a system in which two optical sensors 20 are attached to an end effector 10 (e.g., needle) for tracking and/or determining a position of the end effector (e.g., needle 10). As seen in FIG. 2B, a first optical sensor 20 may be arranged on a distal tip of the needle 10 while a second optical sensor 20 may be proximal to the first optical sensor 20 (e.g., arranged on an elongate member of the needle 10). Accordingly, the first and second optical sensors 20 may be configured to receive acoustic beacon signals generated by a probe (e.g., probe 100 in FIG. 1). The first and second optical sensors 20 (e.g., first optical sensor at the distal tip and the second optical sensor on the elongate member) may be coupled to the same waveguide 22 (e.g., optical fiber, photonic integrated circuit waveguide) to transmit (e.g., propagate) the optical signals to a processing system (e.g., processing system 200 in FIG. 1). The processing system may be configured to generate a first object indicator representative of a position of the tip of the needle 10 (e.g., where the first optical sensor is located) based on the optical signals received from the first optical sensor 20 and a second object indicator representative of a position of the elongate member of the needle 10 (e.g., where the second optical sensor is located) based on the optical signals received from the second optical sensor 20. Additionally or alternatively, the processing system may be configured to generate a single object indicator based on both a position of the tip of the needle 10 and a position of the elongate member using the first and second optical sensors. For example, the object indicator may comprise a vector. Accordingly, a needle 10 may be reliably visualized and tracked during a medical procedure by visualizing and tracking the tip and/or elongate member of the needle 10.

Although FIG. 2A illustrates a single optical sensor 20 for visualizing and tracking an end effector 10 and FIG. 2B illustrates two optical sensors 20 for visualizing and tracking the end effector 10, it should be readily understood that any suitable number of optical sensors may be used to visualize and track an end effector (e.g., three or more optical sensors, such as three, four, five, or more optical sensors). These optical sensors may be attached to, coupled to, integrated with, or otherwise mounted on any suitable part of an end effector. For example, using three optical sensors on a single needle 10 (e.g., one at the needle tip, and two along the elongate member of the needle) may facilitate tracking of a bend of the needle 10 in addition to visualizing and tracking the position of the needle tip. As discussed above, the system 101 in FIG. 1 is described and depicts needle tracking solely for illustrative purposes. It should be readily understood that any other object (e.g., end effector, catheter, guidewire, endoscope, trocar, implant) may be visualized and/or tracked using the systems and methods described herein.

### Exemplary Methods

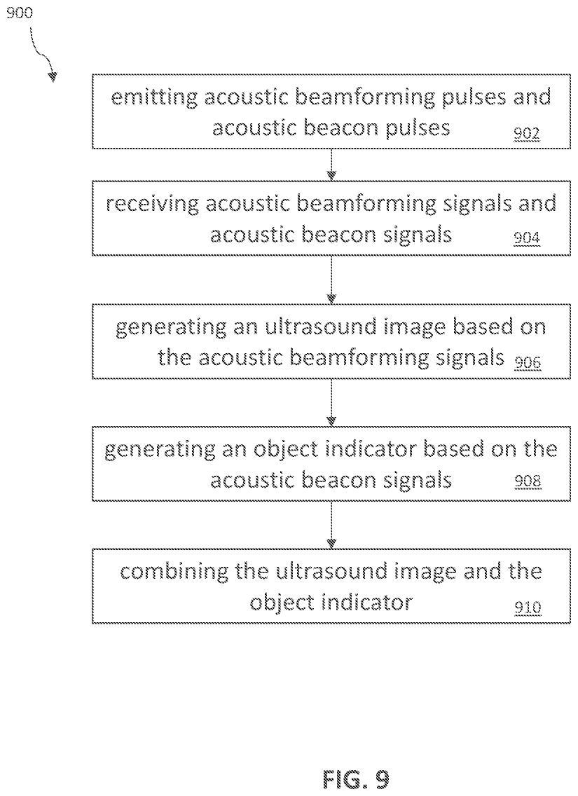

FIG. 9 is a flow diagram illustrating an exemplary variation of a method 900 for object tracking and visualization. At 902, the method 900 may include emitting acoustic beamforming pulses and acoustic beacon pulses. The acoustic beamforming pulses and acoustic beacon pulses may be emitted by a probe (e.g., probe 100 in FIG. 1) including an array of elements (e.g., 1.5D ultrasound array) inclusive of any of the arrays as described herein. In some variations, two or more elements of the array may emit acoustic beacon pulses. For example, two or more elements of the array may be excited using electrical signals generated by a processing system (e.g., processing system 200 in FIG. 1) and configured to emit a set of acoustic beacon pulses. In some variations, one or more of these elements may be configured to emit acoustic beacon pulses independently at different frequencies. Additionally or alternatively, one or more of these elements may be configured to emit the set of acoustic beacon pulses at the same frequency.

In some variations, one or more of the elements may be configured to emit acoustic beacon pulses sequentially. For example, if there are three beacon elements in an array that are configured to emit acoustic beacon pulses, then the first beacon element may be configured to emit a first acoustic beacon pulse at a first time, a second beacon element may be configured to emit a second acoustic beacon pulse at a second time, and a third beacon element may be configured to emit a third acoustic beacon pulse at a third time. In some variations, the first, second, and third beacon elements may be arranged to form a triangle. In some variations, the elements may be excited by an electrical signal at different times to emit the individual acoustic beacon pulses. These acoustic beacon pulses may be emitted periodically and/or sequentially. For instance, acoustic beacon pulses may be emitted at regular or irregular intervals sequentially. Additionally or alternatively, the beacon elements may be configured to emit acoustic beacon pulses substantially simultaneously. In such variations, reflected acoustic beacon signals corresponding to the emitted acoustic beacon pulses may be differentiated as further described below.

In some variations, at least two elements of the plurality of elements configured to emit acoustic beacon pulses may be offset (e.g., spaced apart) from each other in a first dimension (e.g., elevation dimension, lateral dimension). In some variations, one or more beacon elements may be configured to solely emit acoustic beacon pulses. One or more beacon elements may be additionally configured to emit acoustic beamforming pulses and/or receive acoustic beamforming signals. In some variations, a set of acoustic beamforming pulses may be emitted at a frequency that is different from a set of acoustic beacon pulses.

At 904, the method 900 may include receiving acoustic beamforming signals that correspond to acoustic beamforming pulses, and receiving acoustic beacon signals that correspond to acoustic beacon pulses. For example, acoustic beacon signals corresponding to acoustic beacon pulses may be received by an optical sensor (e.g., optical sensor 20 in FIG. 1). The optical sensor may be configured to transmit the optical signals via an optical fiber or other waveguide to a processing system as described herein. Acoustic beamforming signals corresponding to acoustic beamforming pulses may be received by the probe. For example, at least one or more elements of an array of the probe may be configured to receive acoustic beamforming signals. A representation of the acoustic beamforming signals may be transmitted to the processing system.

At 906, the method 900 may include generating an ultrasound image based on the acoustic beamforming signals. In some variations, one or more elements configured to emit acoustic beacon pulses may additionally be configured to emit acoustic beamforming pulses and/or receive acoustic beamforming signals. Therefore, such elements may also contribute to ultrasound image generation. More specifically, such elements may contribute to both object indicator and ultrasound image generation.

At 908, the method 900 may include generating an object indicator based on acoustic beacon signals. As discussed above, in some variations, the elements configured to emit acoustic beacon pulses may do so individually and/or sequentially. In such variations, beacon signals corresponding to the acoustic beacon pulses may be detected sequentially by one or more optical sensors. For instance, in the example considered above, the first beacon element may be configured to emit a first beacon pulse at a first time, a second beacon element may be configured to emit a second beacon pulse at a second time after the first time, and a third beacon element may be configured to emit a third beacon pulse at a third time after the second time. A duration of the beacon pulses may be the same or different. An optical sensor may be configured to detect a first beacon signal corresponding to the first beacon pulse. After the optical sensor detects the first beacon signal, the second beacon element may be configured to emit the second beacon pulse at the second time. The optical sensor may be configured to detect a second beacon signal that corresponds to the second beacon pulse. After the optical sensor detects the second beacon signal, the third beacon element may be configured to emit the third beacon pulse at the third time. The optical sensor may be configured to detect a third beacon signal corresponding to the third acoustic pulse. In this manner, the location of the object may be tracked by emitting the acoustic beacon pulses and detecting the acoustic beacon signals individually and/or sequentially. The processing system may be configured to determine a position of one or more of the optical sensors based on the acoustic beacon signals and generate a corresponding object indicator.

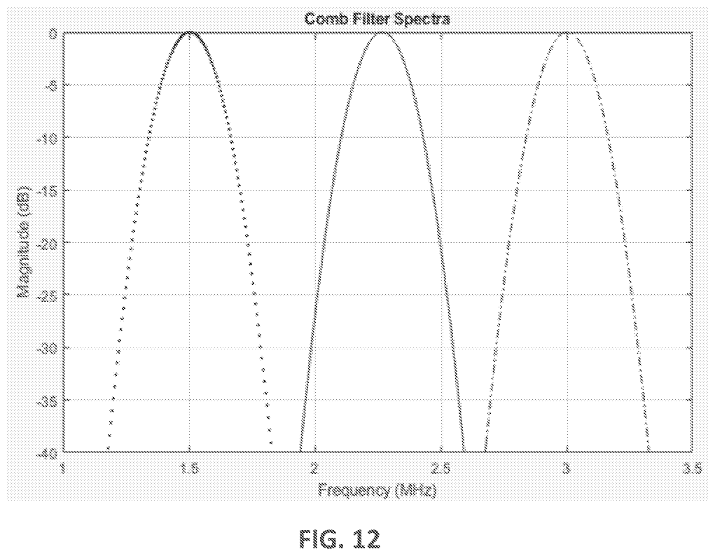

Alternatively, as discussed herein, the beacon elements configured to emit acoustic beacon pulses may do so substantially simultaneously. In such variations, detected acoustic beacon signals may be differentiated in various ways. For example, in one approach, each of the beacon elements may be excited in a manner such that each beacon element emits a respective acoustic beacon pulse at a different frequency. For example, if there are three beacon elements in an array that are configured to emit acoustic beacon pulses, the elements may be excited such that a first beacon element emits a first acoustic beacon pulse at a first frequency, a second beacon element emits a second acoustic beacon pulse at a second frequency, and a third beacon element emits a third acoustic beacon pulse at a third frequency, where the first, second and third frequencies are different. In some variations, the first, second, and third acoustic beacon pulses may be emitted simultaneously. One or more optical sensors may be configured to detect the beacon signals corresponding to the beacon pulses in parallel, and the detected acoustic beacon signals may be separated or distinguished from one another using one or more suitable filters such as a comb filter having center frequencies that correspond to the different frequencies of the acoustic beacon pulses. As such, the comb filter may be configured to filter the detected acoustic beacon signals into a first acoustic beacon signal corresponding to the first acoustic beacon pulse, a second acoustic beacon signal corresponding to the second acoustic beacon pulse, and a third acoustic beacon signal corresponding to the third acoustic beacon pulse. FIG. 12 illustrates an exemplary variation of a comb filter with three distinct frequency ranges. For example, the first range has a center around 1.5 MHz, the second range has a center around 2.25 MHz, and the third range has a center around 3 MHz. The comb filter may be configured to separate a set of acoustic beacon signals having center frequencies around 1.5 MHz, 2.25 MHz, and 3 MHz. In some variations, the processing system may be configured to determine a position of one or more of the optical sensors based on the filtered acoustic beacon signals (e.g., as described below), and generate a corresponding object indicator.

In some variations, differentiating acoustic beacon signals may include exciting each of the elements configured to emit the acoustic beacon pulse with a different coded excitation parameter. Coded excitation parameters may include, for example, parameters that form orthogonal code pairs, such as orthogonal Golay code pairs. In some variations, one or more optical sensors may be configured to detect the beacon signals corresponding to the beacon pulses simultaneously, and a suitable matched filter may be configured to decode the received beacon signals into a first acoustic beacon signal corresponding to the first acoustic beacon pulse, a second acoustic beacon signal corresponding to the second acoustic beacon pulse, and a third acoustic beacon signal corresponding to the third acoustic beacon pulse based on the coded parameters. In some variations, the matched filter may, for example, correspond to the coded excitation parameters. As such, as shown in FIG. 6, in some variations the waveform generator 240 (which generates excitation signals for substantially simultaneously exciting the beacon elements) may be configured to provide data to the signal processor 250 for decoding the acoustic beacon signals into discrete acoustic beacon signals via a filter matched to the coded excitation. In some variations, the processing system may be configured to determine a position of one or more optical sensors based on the acoustic beacon signals and generate a corresponding object indicator.

Additionally or alternatively, coded excitation parameters may include one or more parameters forming Barker code, parameters forming chirp code, parameters forming windowed nonlinear frequency modulation code, combinations thereof, and the like. A suitable matched filter corresponding to coded excitation parameters may be used to decode the received beacon signals as described herein. In some variations, coded excitation parameters may provide a higher signal-to-noise ratio and improved detection accuracy. As an example, one or more beacon elements may be configured to be excited with windowed nonlinear frequency modulation code parameters, as described in further detail in International Patent App. No. PCT/US2022/018515, which is incorporated herein by this reference.

In some variations, the received acoustic beacon signals may be used in a triangulation approach to determine a position of one or more of the optical sensors arranged on an object. FIG. 5 is a schematic illustrating example positions of beacon elements 122 configured to emit acoustic beacon pulses and an example position of an optical sensor 20 in a Cartesian coordinate system. The optical sensor 20 may be arranged on an object (not shown) to be tracked. The location of the object may be determined using the Cartesian coordinate system as described in the example below. In FIG. 5, three beacon elements 122 may be configured to emit acoustic beacon pulses. The beacon elements 122 may form an array (e.g., 1.5D ultrasound array) of a probe (e.g., probe 100). The probe may be configured to emit acoustic beamforming pulses and acoustic beacon pulses (e.g., using elements 122 in FIG. 5) and receive acoustic beamforming signals. Optical sensor 20 may be configured to detect acoustic beacon signals corresponding to the acoustic beacon pulses.

In FIG. 5, the three beacon elements 122 are located at P₁: (−a, 0, 0), P₂: (a, 0, 0), P₃: (0, b, 0), and the optical sensor is located at P: (x, y, z). The distances between the three elements 122 and the sensor 20 may be calculated using the following equations:

$$r 1 = {( {( x + a )}^{2} + {y}^{2} + {z}^{2} )}^{1 / 2} \qquad eqn . ( 1 )$$

$$r 2 = {( {( x - a )}^{2} + {y}^{2} + {z}^{2} )}^{1 / 2} \qquad eqn . ( 2 )$$

$$r 3 = {( {x}^{2} + {( y - b )}^{2} + {z}^{2} )}^{1 / 2} \qquad eqn . ( 3 )$$

Solving Equation 1 and Equation 2 simultaneously results in:

$$x = ( {r}_{1}^{2} - {r}_{2}^{2} ) / 4 a \qquad eqn . ( 4 )$$

Equation 4 indicates that a≠0. That is, the distance between the first element and the second element cannot be zero. Solving Equation 1 and Equation 3 simultaneously results in:

$$y = ( {r}_{1}^{2} - {r}_{3}^{2} - {a}^{2} + {b}^{2} - 2 ax ) / 2 b \qquad eqn . ( 5 )$$

x in Equation 5 may be determined from Equation 4. Eq. 5 indicates that b≠0. That is, the third element cannot be on the line determined by the first element and the second element. For example, the first, second, and third elements may form a triangle. Accordingly, the third element is offset in a first dimension (e.g., elevation dimension). Therefore, from Equation 1:

$$z = {( {r}_{1}^{2} - {( x + a )}^{2} - {y}^{2} )}^{1 / 2} \qquad eqn . ( 6 )$$

where x and y are determined from Equation 4 and Equation 5.

If the acoustic velocity is c and the time required for an acoustic beacon pulse to travel from the first element to the optical sensor is t₁, then:

$${r}_{1} = c {t}_{1} \qquad eqn . ( 7 )$$

r₂ and r₃ may be determined in a similar manner as r₁. Therefore, the location of the optical sensor 20 may be determined based on the time required for an acoustic beacon pulse to travel from an element 122 to the optical sensor 20.

Although the location of the optical sensor 20 may be determined by detecting acoustic beacon signals (e.g., echoes) corresponding to acoustic beacon signal pulses from three beacon elements 122, in some variations, more than three elements 122 may be used to determine the location of the optical sensor. The elements 122 may be positioned in any suitable manner. However, in such a triangulation technique, all of the elements cannot be on a single straight line (e.g., at least one element is offset along a different dimension). For example, a first and second element may be arranged along a lateral dimension and a third element may be arranged along an elevation dimension transverse to the lateral dimension where the third element does not intersect the lateral dimension (e.g., so as to be arranged as vertices of a triangle). Accordingly, the third element in this example is not aligned with respect to the lateral dimension of the first and second elements. The first and second elements are offset with respect to each other but are aligned in the lateral dimension. In some variations, using more than three elements 122 may improve the accuracy of the determined location of the optical sensor 20. In some variations, more than one optical sensor 20 may be used to detect acoustic beacon signals. The position of each optical sensor may be determined similar to as described above.

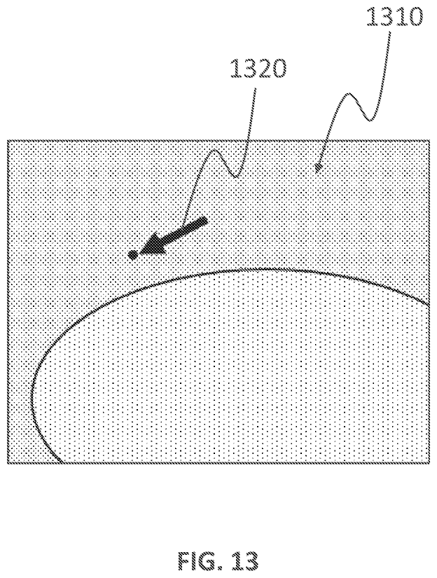

At 910, the method 900 may include combining or otherwise merging the ultrasound image and the object indicator. In some variations, one or more object indicators may be overlaid on the ultrasound images. For example, the schematic of FIG. 13 illustrates a combined ultrasound image 1310 and example variation of an object indicator 1320. The object indicator 1320 shown in FIG. 13 may comprise a graphic icon in the form of an arrow directed toward an object (e.g., needle) to be tracked. However, the object indicator may have any suitable form suitable for indicating the precise location of the object as determined by processing the acoustic beacon signals. For example, the object indicator may be colored and/or animated (e.g., flashing) in a predetermined manner to communicate additional information regarding the location of the object (e.g., proximity to imaging plane) and/or increase visibility of the object indicator. In some variations, the object indicator may comprise one or more visual, audio, and haptic indicators. For example, an audio notification may be output when the object indicator reaches a predetermined location or is within a predetermined distance from a predetermined object (e.g., tissue).

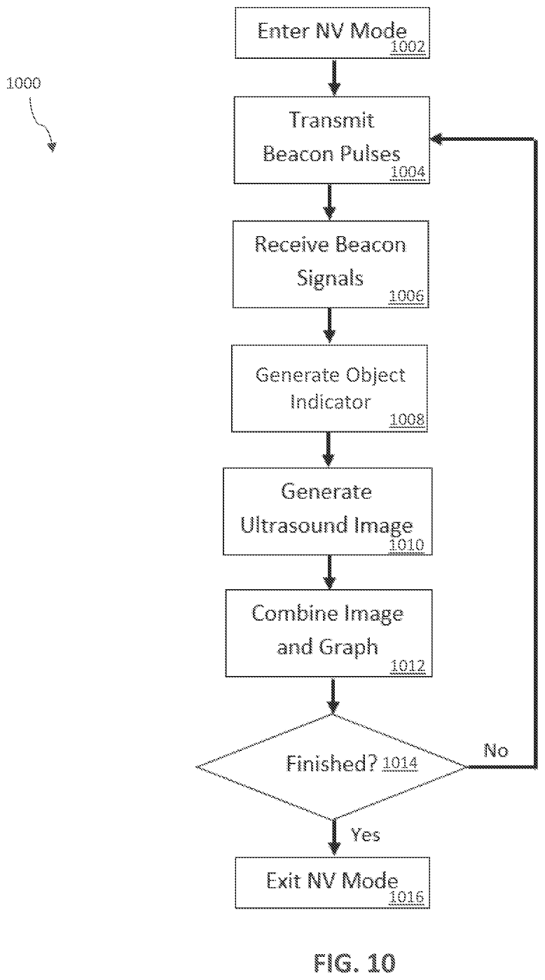

The technology disclosed herein may support different imaging modes such as brightness mode (B-mode), Harmonic Imaging mode, Color Doppler mode, Pulsed-Waved mode (PW mode), and Continuous wave mode (CW mode). FIG. 10 is a flow diagram illustrating an exemplary variation of a needle visualization mode (NV mode) of operation, though it should be understood that another mode could equivalently be used to visualize other objects. After entering the needle visualization mode 1002, the method 1000 may include emitting (e.g., transmitting) acoustic beacon pulses 1004 as described above. In some variations, the acoustic beacon pulses may be emitted by exciting a set of elements in an array of a probe (e.g., probe 100 in FIG. 1) configured to emit acoustic beacon pulses. In some variations, the method 1000 may include receiving acoustic beacon signals 1006 corresponding to the acoustic beacon pulses. For example, an optical sensor may be configured to detect the acoustic beacon signals and transmit the acoustic beacon signals to a processing system (e.g., processing system 200 in FIG. 1).

The method 1000 may include determining a position of the needle using the received acoustic beacon signal and generating an object indicator 1008. In some variations, the method 1000 may switch to another mode such as a B-mode to generate ultrasound images. For example, the method 1000 may include generating ultrasound images 1010 based on received beamforming signals. In some variations, the method may further include combining the ultrasound images and the object indicator (e.g., graph) before displaying them to a user (e.g., display mode) 1012. If the needle visualization mode is not terminated (1014—No), the method 1000 may continue to transmit acoustic beacon pulses 1004. Else, the method 1000 may include exiting the needle visualization mode 1016.

In some variations, the needle visualization mode may include a frame-based interleaf operation mode. For example, the ultrasound image data acquisition and the needle visualization data acquisition may be performed to alternately generate one or more frames of an ultrasound image and one or more frames relating to object tracking (e.g., generation of an object indicator). The interleaf modes may occur in any suitable manner. For example, for each needle visualization data acquisition, two or more ultrasound frame image acquisitions may occur. Additionally or alternatively, needle visualization may include a line-based interleaf operation mode. For example, the ultrasound image data acquisition and the needle visualization data acquisition may be performed alternately to generate one or more lines of a frame of an ultrasound image and one or more lines of a frame relating to object tracking.

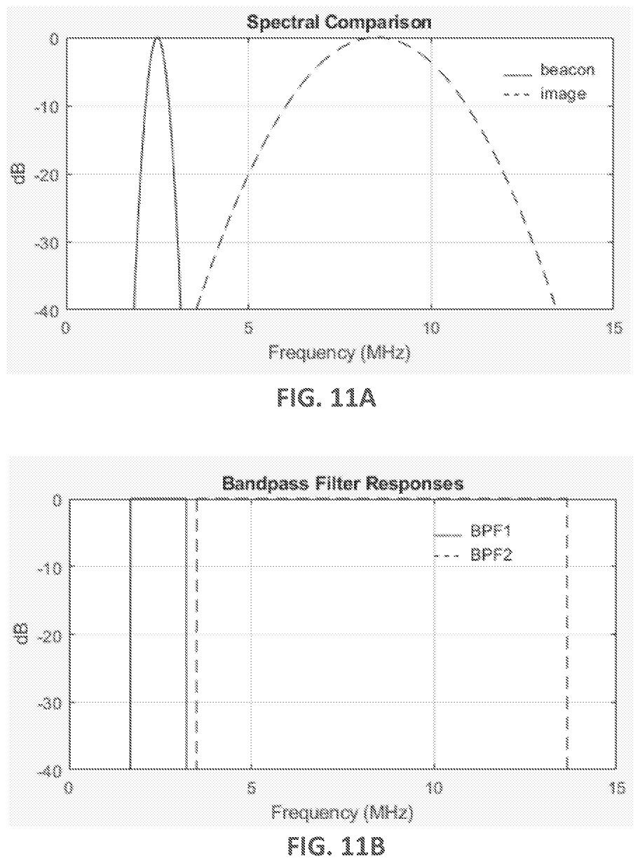

In some variations, needle visualization data and image data may be generated at the same time if the acoustic beacon signals and the acoustic beamforming signals are separated such as with a filter. FIG. 11A illustrate two spectra where the solid lines represent an acoustic beacon signal spectrum and the dashed lines represent an acoustic beamforming signal spectrum. The two spectra have no overlap and may be separated by two filters (e.g., bandpass filter BPF1 and bandpass filter BPF2) with different frequency bands as shown in FIG. 11B. Although bandpass filters are shown in FIG. 11B, any suitable filter may be used to separate the two spectra.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. A method for visualizing position of an object, the method comprising:
emitting acoustic beamforming pulses and acoustic beacon pulses from an ultrasound array, wherein the ultrasound array comprises two or more transducers offset in a first dimension of the ultrasound array;
receiving acoustic beamforming signals corresponding to the acoustic beamforming pulses and acoustic beacon signals corresponding to the acoustic beacon pulses with one or more optical sensors arranged on the object;
generating an ultrasound image based on the acoustic beamforming signals; and
generating an object indicator based on the acoustic beacon signals.

2. The method of claim 1, wherein emitting the acoustic beacon pulses comprises emitting a first acoustic beacon pulse from a first transducer and emitting a second acoustic beacon pulse from a second transducer.

3. The method of claim 2, wherein the second transducer is offset from the first transducer in the first dimension of the ultrasound array.

4. The method of claim 2, wherein receiving the acoustic beacon signals comprises receiving a first acoustic signal corresponding to the first acoustic beacon pulse and a second acoustic signal corresponding to the second acoustic beacon pulse with a single optical sensor of the one or more optical sensors.

5. The method of claim 1, further comprising receiving the acoustic beamforming signals corresponding to the beamforming pulses with at least one transducer.

6. The method of claim 2, wherein emitting the acoustic beacon pulses further comprises emitting a third acoustic beacon pulse from a third transducer.

7. The method of claim 2, comprising emitting the first acoustic beacon pulse from the first transducer at a first time and emitting the second acoustic beacon pulse from the second transducer at a second time subsequent to the first time.

8. The method of claim 2, comprising substantially simultaneously emitting the first acoustic beacon pulse from the first transducer and emitting the second acoustic beacon pulse from the second transducer.

9. The method of claim 8, wherein the first acoustic beacon pulse has a first transmit frequency and the second acoustic beacon pulse has a second transmit frequency different from the first transmit frequency.

10. The method of claim 9, wherein generating the object indicator comprises:
filtering the received acoustic beacon signals into a first acoustic beacon signal corresponding to the first acoustic beacon pulse based on the first transmit frequency; and
filtering the received acoustic beacon signals into a second acoustic beacon signal corresponding to the second acoustic pulse based on the second transmit frequency.

11. The method of claim 10, wherein filtering the received acoustic beacon signals into the first and second acoustic beacon signals comprises applying to the received acoustic beacon signals a comb filter having a first filtering band centered around the first transmit frequency and a second filtering band centered around the second transmit frequency.

12. The method of claim 8, further comprising exciting the first and second transducers with different coded excitation parameters.

13. The method of claim 12, wherein generating the object indicator comprises applying a matched filter to decode the received acoustic beacon signals into a first acoustic beacon signal corresponding to the first acoustic beacon pulse and a second acoustic beacon signal corresponding to the second acoustic beacon pulse.

14. The method of claim 12, wherein the coded excitation parameters comprise parameters forming orthogonal code pairs.

15. The method of claim 14, where the orthogonal code pairs are orthogonal Golay code pairs.

16. The method of claim 12, wherein the coded excitation parameters comprise parameters forming Barker code.

17. The method of claim 12, wherein the coded excitation parameters comprise parameters forming chirp code.

18. The method of claim 12, wherein the coded excitation parameters comprise parameters forming windowed nonlinear frequency modulation code.

19. The method of claim 1, comprising alternating between emitting acoustic beamforming pulses and emitting acoustic beacon pulses.

20. The method of claim 19, comprising alternating between generating an ultrasound image based on the acoustic beamforming signals and generating the object indicator based on the acoustic beacon signals.

21. The method of claim 1, comprising substantially simultaneously emitting the acoustic beamforming pulses and the acoustic beacon pulses.

22. The method of claim 21, wherein the acoustic beamforming pulses have a third transmit frequency and the acoustic beacon pulses have a fourth transmit frequency different from the third frequency.

23. The method of claim 22, further comprising filtering the received acoustic beamforming signals based on the third transmit frequency, and filtering the received acoustic beacon signals based on the fourth transmit frequency.

24. The method of claim 1, wherein generating the object indicator comprises resolving the received acoustic beacon signals into a current object position.

25. The method of claim 1, further comprising combining the ultrasound image and the object indicator.

26. The method of claim 1, wherein the one or more optical sensors comprises an interference-based optical sensor.

27. The method of claim 26, wherein the one or more optical sensors comprises an optical resonator or an optical interferometer.

28. The method of claim 27, wherein the one or more optical sensors comprises a whispering gallery mode (WGM) resonator.

29. The method of claim 1, wherein the one or more transducers comprises one or more of a piezoelectric sensor, a single crystal material sensor, a piezoelectric micromachined ultrasound transducer (PMUT) sensor, and a capacitive micromachined ultrasonic transducer (CMUT) sensor.

30. The method of claim 1, wherein the first dimension is an elevation dimension of the ultrasound array.

31. The method of claim 1, wherein the first dimension is a lateral dimension of the ultrasound array.

32. A system for visualizing position of an object, comprising:
an ultrasound array comprising:
a plurality of transducers configured to emit acoustic beamforming pulses and acoustic beacon pulses, wherein the plurality of transducers comprises two or more transducers offset in a first dimension of the ultrasound array;
at least one optical sensor arranged on the object and configured to detect acoustic beamforming signals corresponding to the acoustic beamforming pulses and acoustic beacon signals corresponding to the acoustic beacon pulses; and
at least one processor configured to generate an ultrasound image based on the acoustic beamforming signals and an object indicator based on the acoustic beacon signals.

33. The system of claim 32, wherein the plurality of transducers comprises a first transducer configured to emit a first acoustic beacon pulse and a second transducer configured to emit a second acoustic beacon pulse, wherein the second transducer is offset from the first transducer in the first dimension of the ultrasound array.

34. The system of claim 33, wherein the plurality of transducers comprises a third transducer configured to emit a third acoustic beacon pulse, wherein a distance between the first transducer and the second transducer in a second dimension of the ultrasound array is different from a distance between the third transducer and the second transducer in the second dimension of the ultrasound array.

35. The system of claim 32, wherein the optical sensor is an interference-based optical sensor.

36. The system of claim 35, wherein the optical sensor is an optical resonator or an optical interferometer.

37. The system of claim 36, wherein the optical sensor is a whispering gallery mode (WGM) resonator.

38. The system of claim 32, wherein the plurality of transducers comprises one or more of a piezoelectric sensor, a single crystal material sensor, a piezoelectric micromachined ultrasound transducer (PMUT) sensor, and a capacitive micromachined ultrasonic transducer (CMUT) sensor.

39. The system of claim 32, wherein the at least one processor is further configured to combine the ultrasound image and the object indicator.

40. The system of claim 32, further comprising a display configured to display one or more of the ultrasound image and the object indicator.

41. The system of claim 32, wherein the ultrasound array is arranged on the object.

42. The system of claim 32, wherein the at least one optical sensor is coupled to the object.

43. The system of claim 32, wherein the at least one optical sensor is integrally formed with the object.

44. The system of claim 43, wherein the object comprises an elongate member and a distal end.

45. The system of claim 44, wherein the at least one optical sensor is arranged on the distal end of the object.

46. The system of claim 44, wherein the at least one optical sensor is arranged on the elongate member.

47. The system of claim 46, wherein the two or more optical sensors are arranged on the elongate member.

48. The system of claim 41, wherein the object comprises a needle.

49. The system of claim 32, wherein the first dimension is an elevation dimension of the ultrasound array.

50. The system of claim 32, wherein the second dimension is a lateral dimension of the ultrasound array.

51. The system of claim 36, wherein the first dimension is transverse to the second dimension.
